(19) United States
(12) Patent Application Publication
(10) Pub. No.: US 2011/0106118 A1
(43) Pub. Date: May 5, 2011

(54) ABSORBABLE VASCULAR ANASTOMOTIC SYSTEM

(75) Inventors: Dae Gu Son, Daegu (KR); Joon Hyung Kim, Cheongwon-gun (KR); Kyo Chang Choi, Cheongwon-gun (KR); Seung Ho Song, Cheongwon-gun (KR)

(73) Assignee: META BIOMED CO., LTD., Cheongwon-gun (KR)

(21) Appl. No.: 12/674,132

(22) PCT Filed: Jul. 24, 2008
(86) PCT No.: PCT/KR08/04335

§ 371 Date: Feb. 18, 2010

(30) Foreign Application Priority Data
Aug. 29, 2007 (KR) 10-2007-0087047

(51) Int. Cl. A61B 17/11

(52) U.S. Cl. 606/153

(57) ABSTRACT

Provided is an absorbable vascular anastomotic system including an insertion tube of which a front end has a plurality of hooks protruding outward and a rear end has ring-shaped protrusions, and an interlocking tube having grooves corresponding to the protrusions of the insertion tube such that two insertion tubes are inserted into the interlocking tube at both ends of the interlocking tube to be fixed. The insertion tube and the interlocking tube are formed of a material which is absorbed into a living body, and when two insertion tubes into which two blood vessels are respectively inserted to be fixed to the hooks are inserted into the interlocking tube at both ends of the interlocking tube, inner walls of both the blood vessels come into contact with each other in the center of the interlocking tube to be anastomosed. Since the absorbable vascular anastomotic system does not require an additional anastomotic device, it is possible to perform the anastomosis even in a narrow space. Further, since the absorbable vascular anastomotic system is completely absorbed into a living body, it is possible to avoid a risk of foreign body reaction. After the absorbable vascular anastomotic system is completely absorbed, the anastomosed vessels recover their own elasticity.

### TECHNICAL FIELD

The present invention relates to a vascular anastomotic system which connects blood vessels in a free flap, and more specifically, to an absorbable vascular anastomotic system which can simply anastomose blood vessels by using two insertion tubes and one interlocking tube.

### BACKGROUND ART

A free flap is used for reconstructing tissue defects which are caused by an external wound or occur after tumor excision. The free flap is an operative technique for transferring a tissue by completely cutting a blood vessel which supplies a blood flow to a donor tissue, transferring the blood vessel to a recipient portion, and then connecting the blood vessel to a blood vessel of the recipient portion through a microvascular anastomosis. In the vascular anastomosis, the most important step of the free flap, a very fine suture is frequently used. In particular, at least one artery and two veins should be connected in the microvascular anastomosis. Although the microvascular anastomosis is performed by an experienced surgeon specializing in microsurgery, it takes about 1 hour 30 minutes to complete, which indicates that the microvascular anastomosis is difficult to perform. Therefore, it requires quite a long time to learn the operative technique. In the microvascular anastomosis, the veins are more difficult to suture than the artery. Further, when a difference in diameter between a donor vessel and a recipient portion is large, or the donor vessel and the recipient portion are disposed perpendicular to each other, the suturing is more difficult to perform.

Further, in the microvascular anastomosis using a suture, a needle may damage the inner wall of the blood vessel while it passes through, thereby causing a complication such as thrombosis. When a difference in diameter between a donor vessel and a recipient portion is large, there are difficulties in applying the microvascular anastomosis. Further, when the flap ischemic time lengthens as the anastomosis time extends, a risk of complication caused by ischemia-reperfusion injury increases.

To overcome such a disadvantage, Nakayama and others developed a microvessel stapler including two metallic rings and twelve interlocking pins, in 1962. Although a few modified microvessel staplers have been developed since then, only the MAC (Microvascular Anastomotic Coupler) system made by Synovis™, Birminham, USA, is currently used in clinics.

Referring to FIGS. 9 to 11, a method for using the MAC system will be described. First, the diameter of a blood vessel is measured by a vessel measuring gauge, and a ring with a proper diameter is selected to be mounted on an anastomotic device. Subsequently, the anastomotic device is placed between a donor vessel and a recipient vessel, and one vessel is inserted into the ring and turned over such that the lining membrane of the vessel is everted. Then, the vessel is fixed to a pin provided on the ring. The other vessel is fixed in the same manner. Next, a turning unit provided at an end of a handle of the anastomotic device is turned to concentrate both of the rings to the center to complete the anastomosis. Finally, when the turning device is further turned, the rings engaged with each other gradually come off from the anastomotic device.

The MAC system has a larger number of advantages than the microvascular anastomosis using a suture. First, the vessel anastomosis using the MAC system takes only about 5 minutes. The short anastomosis time reduces flap ischemia, which makes it possible to reduce a risk of complication caused by ischemia-reperfusion injury and to save overall operation time. Therefore, the MAC system can be effectively used in an operation which requires a long time, like an operation where two free flaps or a vein graft is needed. Second, even when a difference in diameter between a donor vessel and a recipient vessel is large, the vessels can be easily anastomosed. Although the diameter of one vessel is 3.5 times larger than that of the other vessel, the vessels can be easily anastomosed. Third, long-term follow-up results after the operation are not inferior to those of the vessel anastomosis using a suture. In a case of an irradiated vessel as well as a normal vessel, the mechanical vessel anastomosis exhibits a similar or superior graftpatency rate to the vessel anastomosis using a suture. Finally, the duration of training required for learning the operative technique is not longer than in the vessel anastomosis using a suture. Therefore, it is possible to increase a success rate.

Although the MAC vascular anastomotic system has many advantages, its price is much higher than the suture. Therefore, the MAC vascular anastomotic system is difficult for patients to afford. Further, since the MAC vascular anastomotic system remains forever in the body, a foreign body reaction may occur. Furthermore, since the vascular anastomotic system is formed of polyethylene, it may apply pressure to surrounding vessels, and it has no elasticity unlike normal vessels.

### DISCLOSURE OF INVENTION

### Technical Problem

The present invention is directed to an absorbable vascular anastomotic system through which a vessel anastomosis can be simply performed, and which is completely absorbed into a living body after a predetermined time, thereby minimizing a foreign body reaction.

### Technical Solution

In one aspect, an absorbable vascular anastomotic system includes: an insertion tube of which a front end has a plurality of hooks protruding outward and a rear end has ring-shaped protrusions; and an interlocking tube having grooves corresponding to the protrusions of the insertion tube such that two insertion tubes are inserted into the interlocking tube at both ends of the interlocking tube to be fixed. Here, the insertion tube and the interlocking tube are formed of a material which is absorbed into a living body, and when two insertion tubes into which two blood vessels are respectively inserted to be fixed to the hooks are inserted into the interlocking tube at both ends of the interlocking tube, inner walls of both the blood vessels come into contact with each other in the center of the interlocking tube to be anastomosed.

The front end of the insertion tube may be a cylindrical tube which is divided into three parts at an interval of 120 degrees, and each of the divided parts may have two hooks disposed thereon.

The internal center portion of the interlocking tube may have such a space as not to come into contact with the hooks of the insertion tube.

The insertion tube and the interlocking tube may be formed of polylactic-glycoilic acid (PLGA), and absorption time may be controlled by adjusting a mixing ratio of lactic acid and glycolic acid.

### Advantageous Effects

Although the conventional MAC system requires an additional space for a separate anastomotic device, the absorbable vascular anastomotic system according to the present invention does not require an additional anastomotic device. Therefore, it is possible to perform the anastomosis even in a narrow space.

Further, while the MAC system permanently remains in a living body and so may cause a foreign body reaction, the absorbable vascular anastomotic system according to the present invention is completely absorbed into a living body and so it is possible to avoid a risk of foreign body reaction. When the absorbable vascular anastomotic system is completely absorbed, the anastomosed vessels recover their own elasticity.

Furthermore, since the absorbable vascular anastomotic system according to the present invention can be produced at a low cost, microsurgeries can be made more accessible to patients.

### BRIEF DESCRIPTION OF THE DRAWINGS

FIGS. 1 to 3 are a perspective view, a side view, and a plan view of an insertion tube according to an example embodiment of the present invention, respectively.

FIG. 4 is a cross-sectional view of an interlocking tube according to the example embodiment of the present invention.

FIG. 5 is a diagram for explaining a state where two insertion tubes are inserted into the interlocking tube.

FIG. 6 shows a state before a blood vessel is inserted into the insertion tube.

FIG. 7 shows a state where the blood vessel is inserted into the insertion tube to be fixed by hooks.

FIG. 8 shows a state where the blood vessel has been anastomosed by the insertion tubes and the interlocking tube.

FIG. 9 is a diagram for explaining a conventional MAC system.

FIGS. 10 and 11 are diagrams for explaining a method for anastomosing vessels by using the conventional MAC system.

### MODE FOR THE INVENTION

Hereinafter, example embodiments of the present invention will be described in detail with reference to the accompanying drawings. However, it will be appreciated by those skilled in the art that various changes may be made to these example embodiments, and the scope of the present invention is not limited to the example embodiments.

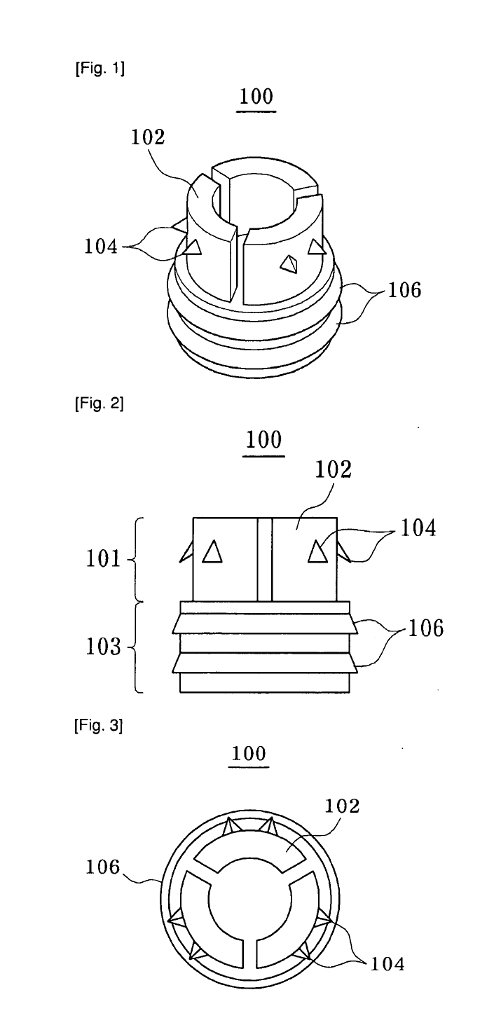

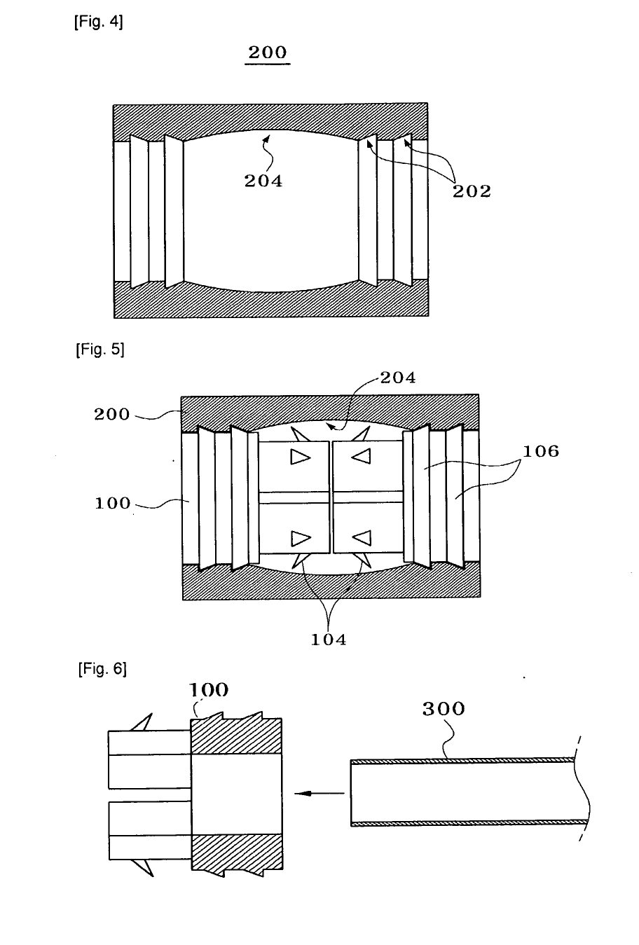

An absorbable vascular anastomotic system according to the present invention includes two insertion tubes and one interlocking tube. The insertion tubes will be described by referring to FIGS. 1 to 3, and the interlocking tube will be described by referring to FIGS. 4 and 5. Then, a method for performing vascular anastomoses by using the insertion tubes and the interlocking tube will be described by referring to FIGS. 6 to 8.

FIGS. 1 to 3 are a perspective view, a side view, and a plan view of an insertion tube 100 according to an example embodiment of the present invention, respectively.

As shown in FIG. 2, the insertion tube 100 according to the example embodiment of the present invention may be divided into front and rear ends 101 and 103. The front end 101 has a plurality of hooks 104 attached on a cylindrical tube represented by reference numeral 102, the hooks 104 protruding outward from the cylindrical tube. The cylindrical tube 102 may be formed of one tube as a whole, or may be divided into three parts at an interval of 120 degrees, as shown in FIG. 1. According to this example embodiment of the present invention, each of the divided parts 102 has two hooks 104 disposed thereon.

The rear end 103 of the insertion tube 100 has a ring-shaped protrusion 106 provided thereon. As shown in FIGS. 1 and 2, two ring-shaped protrusions 106 may be disposed on one insertion tube. According to an example embodiment, the total length of the insertion tube 100 including the front and rear ends 101 and 103 is set to about 4 mm, the outer radius of the cylindrical tube is set to 2.25 mm, and the height of the hook is set to about 0.5 mm. The dimensions correspond to the size of a vascular anastomotic system for anastomosing a blood vessel with a diameter of 2-3 mm, which is most frequently anastomosed in micro surgeries. The size of the vascular anastomotic system can be adjusted depending on changes in the diameter of blood vessels.

FIG. 4 is a cross-sectional view of an interlocking tube 200 according to the example embodiment of the present invention. FIG. 5 is a diagram for explaining a state where two insertion tubes 100 are inserted into the interlocking tube 200.

Referring to FIGS. 4 and 5, the interlocking tube 200 has a structure whereby two insertion tubes 100 can be inserted into the interlocking tube 200. Therefore, the inside of the interlocking tube 200 is formed in a cylindrical shape, and the interlocking tube 200 has grooves 202 corresponding to the ring-shaped protrusions 106 of the insertion tubes 100. Since the interlocking tube 200 has a structure whereby two insertion tubes 100 can be inserted into the interlocking tube 200 from both ends, the interlocking tube 200 has two grooves 202 provided at either end thereof. The interlocking tube 200 should have a space 204 formed in the inner central portion thereof, the space 204 being large so that the inner central portion of the interlocking tube 200 does not come in contact with the hooks.

According to an example embodiment, the length of the interlocking tube 200 is set to 8.1 mm, and the outer radius of the interlocking tube 200 is set to 3 mm.

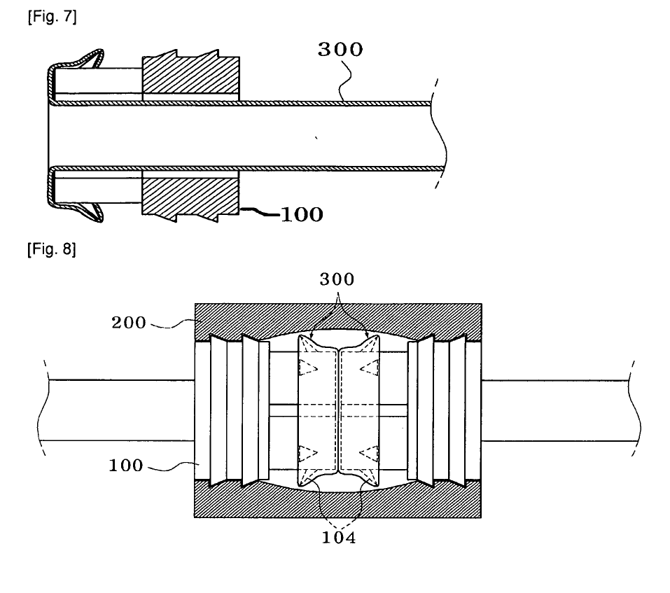

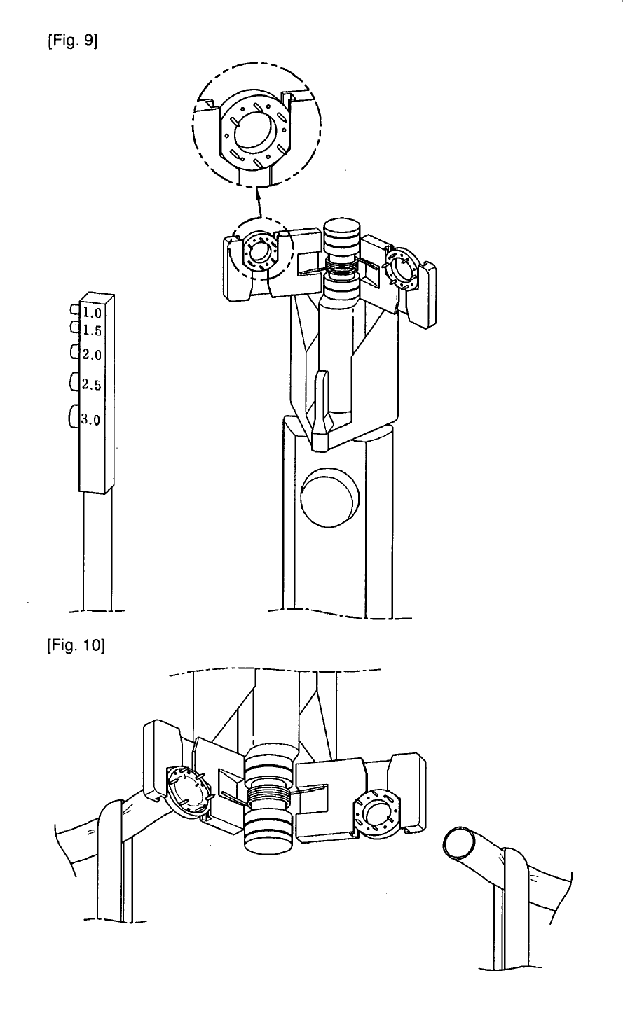

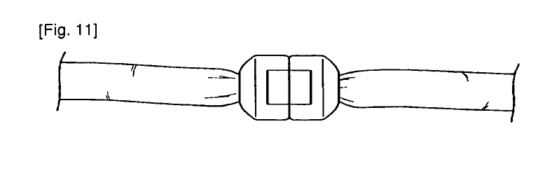

FIG. 6 shows a state before a blood vessel 300 is inserted into the insertion tube 100. FIG. 7 shows a state where the blood vessel 300 is inserted into the insertion tube 100 to be fixed by the hooks. FIG. 8 shows a state where the blood vessel 300 has been anastomosed by the insertion tubes 100 and the interlocking tube 200.

Referring to FIG. 6, the blood vessel 300 is inserted into the insertion tube 100 in order to be anastomosed. Referring to FIG. 7, the blood vessel 300 is passed through the insertion tube 100 and then turned over to be fixed to the hooks 104. Another blood vessel positioned on the opposite side is inserted into the insertion tube in the same way.

Referring to FIG. 8, the insertion tubes 100 into which the blood vessels 300 are respectively inserted are inserted into both ends of the interlocking tube 200. Then, the insertion tubes 100, into which two blood vessels are respectively inserted, are fixed to the interlocking tube 200 through the ring-shaped protrusions 106 of the insertion tubes 100, and join each other at the center of the interlocking tube 200. The inner walls of both the blood vessels come in contact with each other, and are then covered with endothelial cells in 4-5 days to be anastomosed.

Therefore, by using the vascular anastomotic system including two insertion tubes and one interlocking tube, the anastomosis can be completed within a short time, and a difference in diameter between two blood vessels can be easily overcome. Further, since the duration of training required for learning the technique is not long, it is possible to achieve a high success rate.

The vascular anastomotic system according to the present invention should be absorbable into a living body. Since it takes only 2-3 weeks from the vascular anastomosis until a gap between the inner surfaces of blood vessels is filled up, the absorbable vascular anastomotic system may be formed of polylactic-glycoilic acid (PLGA) to be absorbed into a living body within several months. In this case, the absorption time may be controlled by adjusting a mixing ratio of lactic acid and glycolic acid.

More specifically, the degradation of biodegradable polymer occurs in intrachain bonds where the biodegradable polymer can be hydrolyzed by microorganisms in the water or soil. As the degradation progresses, the molecular weight of the biodegradable polymer decreases. Finally, the biodegradable polymer is recovered as a monomer, or degrades into water and carbon dioxide.

As for the hydrolysable intrachain, amide, ester, urea, urethane and so on are well known. Among them, aliphatic polyester has important physical and chemical properties, exhibits sufficient degradation, and is obtained from microorganisms or chemical synthesis. The aliphatic polyester is classified into two groups. One group includes Poly Lactide (PLA), Polyglycolide (PGA), Poly Caprolactone (PCL) and so on, which are obtained from chemical synthesis, and the other group includes Poly Hydroxybutyrate (PHB), Poly Hydroxybutyrate-co-Valerate (HB-co-HV) and so on, which are obtained from microorganisms.

Among them, PLA is widely used as a biomedical material, because it exhibits biodegradation, bio-compatibility, excellent mechanical properties, and is easily dissolved in a general solvent during a process. However, since PLA has low biodegradation speed, it has a limit in specific biomedical use. Therefore, as glycolide is introduced into polymer chains through copolymerization, it is possible to control the degradation speed. PLGA, a copolymer of PLA and PGA, exhibits different degradation speeds depending on the ratio of LA to GA in the copolymer. When the ratio of LA to GA is 50:50, the degradation speed is the highest. In this case, PLGA completely degrades in about two months.

1. An absorbable vascular anastomotic system comprising:
an insertion tube of which a front end has a plurality of hooks protruding outward and a rear end has ring-shaped protrusions; and
an interlocking tube having grooves corresponding to the protrusions of the insertion tube such that two insertion tubes are inserted into the interlocking tube at both ends of the interlocking tube to be fixed,
wherein the insertion tube and the interlocking tube are formed of a material which is absorbed into a living body, and
when two insertion tubes into which two blood vessels are respectively inserted to be fixed to the hooks are inserted into the interlocking tube at both ends of the interlocking tube, inner walls of both the blood vessels come into contact with each other in the center of the interlocking tube to be anastomosed.

2. The absorbable vascular anastomotic system of claim 1, wherein the front end of the insertion tube is a cylindrical tube which is divided into three parts at an interval of 120 degrees, and each of the divided parts has two hooks disposed thereon.

3. The absorbable vascular anastomotic system of claim 1, wherein the internal center portion of the interlocking tube has such a space as not to come into contact with the hooks of the insertion tube.

4. The absorbable vascular anastomotic system of claim 1, wherein the insertion tube and the interlocking tube are formed of polylactic-glycoilic acid (PLGA), and absorption time is controlled by adjusting a mixing ratio of lactic acid and glycolic acid.
